(19) United States
(12) Patent Application Publication
(10) Pub. No.: US 2003/0082597 A1
(43) Pub. Date: May 1, 2003

(54) Age-associated markers

(76) Inventors: L. Edward Cannon, Cambridge, MA (US); Cynthia A. Bayley, Norwell, MA (US); Cynthia J. Kenyon, San Francisco, CA (US); Leonard P. Guarente, Chestnut Hill, MA (US); Alan D. Watson, Lexington, MA (US)

Correspondence Address: FISH & RICHARDSON PC, 225 FRANKLIN ST, BOSTON, MA, 02110, US

(21) Appl. No.: 10/219,443

(22) Filed: Aug. 15, 2002

Related U.S. Application Data
(60) Provisional application No. 60/312,734, filed on Aug. 15, 2001.

(51) Int. Cl. C12Q 1/68; G01N 33/53

(52) U.S. Cl. 435/6; 435/7.1

(57) ABSTRACT

Disclosed is a method of identifying an biological age-associated marker. The method can include: providing a first organism having a first genotype and a second organism having a second genotype, wherein the first and second organisms are derived from the same species and are the same chronological age; and comparing a property associated with a biomolecule in the first organism to a property associated with the biomolecule in the second organism to identify a biomolecule having a preselected value for said property, thereby identifying the biomolecule as an biological age-associated marker.

### CROSS-REFERENCE TO RELATED APPLICATIONS

This application claims priority to U.S. Application Serial No. 60/312,734, filed on Aug. 15, 2001, the contents of which is incorporate by reference in its entirety for all purposes.

### BACKGROUND

Numerous processes in biology are conserved. For example, although the body plan organization of mammals and fruit flies and nematodes bear little visual resemblance, the fundamental molecular controls of body plan organization are highly conserved. For example, all these organisms include clusters of genes encoding homeobox proteins that specify cell identify in the body plan (Kenyon et al. Trends Genet. 1994;10(5):159-64).

Conservation among diverse animals also extends to molecular mechanisms of lifespan regulation—despite great disparity in expected lifespan. In one example, the levels of insulin-like growth factor regulate the lifespan of at least both nematodes and mice. In the nematode C. elegans, mutations of the daf-2 gene, which encodes an insulin-like growth factor receptor, extends lifespan at least 50% (Kenyon et al. Nature 1993; 366:461-4). This lifespan extension phenotype in nematodes is dependent on the HNF-3/forkhead transcription factor daf-16. In mice, the levels of insulin-like growth factor are also correlated with lifespan. Ames mice which have extended lifespan and a homozygous mutation of the Prop-1 gene are characterized by the near absence of growth hormone producing cells, and consequently reduced insulin-like growth factor-1 (IGF-1) (Brown-Borg Nature 1996; 384:33). In addition, the proteins such as the insulin-like growth factor receptor and transcription factor proteins are conserved at the amino acid sequence level among nematodes and mammals.

Other processes have been found to impact the rate of physiological aging. These processes include responses to oxidative damage, regulation of gene silencing, and metabolic sensing (Guarente and Kenyon, Nature 2000; 408:255). Many phenotypic aspects of aging are also similar between disparate animals. The appearance of older animals also typically differs from younger animals.

There is a need to better identify and quantify biological indicators or markers, of aging. For example, such indicators and markers can be used to evaluate biological aging of an individual. Since biological age can differ from chronological age and may vary widely among individuals and circumstance, markers that are correlated with a particular biological age can be used to more accurately and objectively evaluate biological age. Understanding biological age is important for many aspects of medicine, pharmacology, sociology, and agriculture, to name but a few relevant fields.

### SUMMARY

The present invention provides, inter alia, a method of identifying an biological age-associated marker.

In one aspect, the invention features a method that includes: providing a first organism having a first genotype and a second organism having a second genotype, wherein the first and second organisms are derived from the same species and are the same chronological age; and comparing a property associated with a biomolecule in the first organism to a property associated with the biomolecule in the second organism to identify a biomolecule having a preselected value for said property, thereby identifying the biomolecule as an biological age-associated marker. Typically the organisms are animals. The marker, for example, can provide an indication of lifespan regulation in organisms derived from the particular species, and may be predictive of the potential lifespan of an individual. Typically, the comparing is repeated for a property of each of a plurality of biomolecules. In such cases, it is possible to identify a plurality of markers, the plurality being a subset of the plurality of biomolecules.

In one embodiment, a plurality of properties associated with the biomolecule is compared.

The comparing can include providing a first biological sample from the first organism and a second biological sample from the second organism and evaluating the property of the biomolecule in the respective biological samples.

Examples of biomolecules include nucleic acids (e.g. DNA, RNA including mRNA, rRNA, snRNA and other untranscribed RNAs, e.g., small interfering RNAs), proteins, polysaccharides, lipids, or metabolites.

In one embodiment, the property is presence or abundance (e.g. molar concentration).

In another embodiment, the property is chemical composition of the biomolecule, e.g. nucleic acid sequence, amino acid sequence, hydrocarbon chain length, or modification state. For example, the property includes a post-translational modification, e.g. phosphorylation, glycosylation, ubiquitination, sulfation, acylation, prenylation, methylation at one or more positions, e.g., in an amino acid sequence. In another embodiment, the property is a functional activity, e.g., enzymatic activity or binding activity. In another example the functional activity is evaluated in the presence of a reactive oxygen species (ROS), e.g., to indicate resistance or sensitivity to the ROS.

The property of the identified biomolecule can be abundance and the pre-selected value can correspond to at least a 1.2, 2, 5, 10 or 50 fold difference in the property. Similar preselected quantitative relationships can be used as criteria in other comparisons.

In another embodiment, the property is subcellular distribution (e.g. ER, Golgi, cytosolic, nuclear, lysosomal, endosomal, plasma membrane) or physical association with another biomolecule. In one embodiment, the biomolecule is an MRNA transcript and the property is exon organization.

Methods of comparing nucleic acids can include analysis of expressed-sequence tags (EST), gene expression, or transcriptional profiles, or nucleic acid tag analysis e.g. Serial Analysis of Gene Expression (SAGE), or subtractive hybridization methods such as differential display of messenger RNA or CDNA copies of messenger RNA. Methods of comparing proteins can include antibody-based assays, mass spectrometric analysis, enzymatic activity assays, and ligand binding assays. Methods of comparing lipids and polysaccharides include mass spectrometry, thin-layer chromatography, antibody-based assays, and chemical sequencing or analysis. Any method can also include an in silico component.

In one embodiment, the comparing includes evaluating the property using a heterologous reporter of the property. In some embodiments, the heterologous reporter is a heterologous reporter gene operably linked to a regulatory region of a gene encoding the biomolecule. Heterologous reporter genes include genes whose expression can be easily detected, for example, by measuring chemiluminescence, fluorescence, antibody binding, or enzymatic activity. Commonly used reporter genes can encode, e.g., a drug resistance protein (e.g., beta-lactamase or chloramphenicol acetyltransferase), a fluorescent protein (e.g., green fluorescent protein), an enzyme (e.g., beta-galactosidase, luciferase, alkaline phosphatase) or tagged proteins.

In one embodiment, the comparing can include evaluating the respective sample to provide a sample profile that includes information about a property for each of a plurality of candidate markers. Information about the profile can be stored in a machine-accessible medium, and the statistical significance of differences between corresponding candidate markers can be evaluated. The information that identifies a subset of the candidate markers for which the differences are statistically significant can be displayed.

The first genotype can be a wildtype genotype, and the second genotype can be a mutant genotype. In one embodiment, the second genotype includes a naturally occurring genetic variation that alters lifespan. In another related embodiment, the second genotype includes a genetic lesion (e.g. the lesion being a point mutation, a deletion, an insertion, a chromosomal rearrangement, transposon insertion, or retroviral insertion). In a preferred embodiment, the genetic lesion causes altered lifespan, e.g., lifespan extension or lifespan reduction. In one embodiment, the second and/or first genotype includes an exogenous nucleic acid, e.g., a transgene.

The second genotype can be homozygous for the genetic lesion. Alternatively, the second genotype can be heterozygous for the genetic lesion. In another embodiment, the second genotype includes mutations in two different genes. In one embodiment, the second genotype includes mutations in the two different genes, for which it is homo- or heterozygous. In another embodiment, the first genotype is a mutant genotype, and the second genotype is also a mutant genotype, e.g., relative to a wildtype genotype. For example, the first genotype causes lifespan extension relative to wildtype organisms of the same species and the second genotype causes lifespan reduction relative to wildtype organisms of the same species. In another example, both genotypes cause lifespan extension, e.g., by perturbing different pathways.

In a preferred embodiment, the chronological age is an adult age, e.g. an age at which a wildtype organism is in a developmentally mature stage, or at a chronological age in which a wildtype organism can reproduce or is fertile. In one embodiment, the chronological age is an age after the age at which the organism stops growing in size (e.g., height), or an age after the age at which the organism reduces or stops cell divisions in particular tissues. In one embodiment, the chronological age of the organism is an age at which a wildtype organism is adult but before the adult shows overt signs of physiological deterioration due to aging.

Exemplary chronological ages can be between 10-30, 30-50, 50-75, 10-75, 75-100, 85-100, or 40-60% of the average lifespan of the first organism, a wildtype organism, or an average organism of the species.

In one embodiment, the second organism has an average lifespan that is at least 5, 10, 20, 40, 50, or 100% greater than the average lifespan of the first organism. In an embodiment, the second organism has an average lifespan that is at least 5, 10, 20, 40, 50, or 100% greater than the average lifespan of wildtype organisms of the same species. In another embodiment, the second organism has an average lifespan that is at least 5, 10, 20, 40, or 50% less than the average lifespan of wildtype organisms of the same species.

In one embodiment, the second genotype is manifest as a defect in a growth hormone or insulin-like growth factor signaling component, e.g. a defect in signaling via: an insulin/IGF-1-like hormone receptor, such as daf-2 or daf-2 homologs, a PI(3) kinase family member such as age-1 and age-1 homologs, pdk-1 and pdk-1 orthologs and homologs, an insulin/IGF-1-like hormone, such as ceinsulin-1 and ceinsulin-1 orthologs and homologs, a Forkhead transcription factor such as daf-16 and daf-16 homologs which include AFX, FKHR, FKHRL1, and a PTEN phosphatase such as daf-18 and daf-18 orthologs and homologs. In an alternate embodiment, the second genotype causes a defect in chromatin silencing. For example, the defect is in histone deacetylation or a pathway that modulates histone deacetylation. Examples of genes for which mutation perturbs modulation of histone deacetylation include Sir2, Sir3, Sir4, Rpd3, and orthologs and homologs of these genes. In another embodiment, the second genotype causes a defect in metabolite sensing or metabolite transport. Examples of genes that are involved in metabolite sensing include the SNF1 kinase, SIP2, a co-repressor of SNF-1, and SNF4, a coactivator of SNF1, clk-1, coq7, NPT1 and orthologs and homologs of these genes. Exemplary transporters include transporters of carboxylates, e.g., dicarboxylates and tricarboxylates, e.g., the Indy transporter and orthologs and homologs thereof. In yet another embodiment, the second genotype causes a defect in genes that regulate response to oxidative stress. Examples of proteins involved in the response to oxidative stress include catalases such as ctl-1, superoxide dismutases such as sod-3, succinate dehydrogenases such as mev-1, signaling adaptor components such as p66shc, spe-10, spe-26, and old-1. In another embodiment, the second genotype causes a defect in genes that involve endocrine signaling. In one example, the gene encodes a component of the growth hormone-IGF-1 signaling axis, e.g., growth hormone, growth hormone receptor, growth hormone releasing hormone, GHRH receptor, pit-1 and prop 1. In another embodiment, the second genotype is caused by a defect in a G-protein-coupled receptor. In a preferred embodiment, the G-protein-coupled receptor is methuselah or an ortholog or homolog of methuselah. In another embodiment, the genotype is caused by a mutation in the tyrosine kinase tkr-1 or a homolog of tkr-1. A homolog can be at least 30, 50, 70, 80, 90, or 95% identical in sequence to the sequence of interest, e.g., in a region of at least 50, 100, or 300 amino acids or nucleotides, typically in a functional domain or a region encoding a functional domain.

In one embodiment, the first and second organisms are congenic or isogenic, but for at least one genetic difference that causes a difference in average expected lifespan. In some cases, the first and second organisms are siblings.

Typically the first and second organisms are maintained under the same (or substantially similar) controlled conditions, e.g., laboratory conditions. In certain embodiments, the conditions include an environmental element which may modulate an aspect of aging. For example, the environmental element may be a stress, e.g., UV light, oxygen radicals, toxins, a particular diet, and so forth. In one embodiment, a marker is select such that its property of interest is unaffected by metabolic intake, e.g., unaffected by caloric restriction (e.g., when genetically similar or identical organisms are compared).

In one embodiment, the comparing is repeated at multiple chronological ages.

The biological samples can include cells, e.g., fixed or live cells. In one embodiment, the biological samples include purified nucleic acids, e.g., a complex sample of nucleic acids that is free of proteins, lipids, and other compounds, e.g., a DNA preparation, an RNA preparation, or a poly-adenylated RNA preparation. In another embodiment, the biological samples include purified proteins, e.g., a complex protein sample that is free of nucleic acids, lipids, and other compounds, e.g., a complex protein preparation, e.g., a chromatographic fraction, precipitate, and so forth. These purified proteins can retain their native three-dimensional structure, or can be denatured.

In a preferred embodiment, the method further includes: selecting, from biomolecules of a second animal species, an ortholog of the identified marker, and evaluating the property of the ortholog in an organism of the second species. The evaluating can include evaluating the property of the ortholog in genetically-identical organisms of the second species, the organisms being of a differing chronological age. The genetically-identical organisms can be wildtype organisms or genetically altered organisms.

In another embodiment, the evaluating includes evaluating a property of the ortholog in a first organism of the second species and a second organism of the second species with a genotype distinct from the first organism of the second species. In a preferred embodiment, the first and second organisms of the second species are of the same chronological age. The second organism of the second species can have an average lifespan at least 5, 10, 20, 50, 100% greater than the average lifespan of the first organism of the second species. In one example, the first species is a non-mammalian species, and the second species is a mammalian species (e.g. a mouse, primate, human, or transgenic mouse containing human genes).

In one aspect, the method further includes evaluating a property of the marker in a third biological sample. In one embodiment, the third biological sample is obtained from a wildtype animal. In another embodiment, the third biological sample is obtained from cells cultured in vitro. For example, the third biological sample is obtained from cultured cells treated with a test compound. In another example, the third biological sample is obtained from an animal treated with a test compound. Most preferably, the treated animal is treated with the test compound for less than 25%, 10%, 5%, 1%, or 0.1% of its average lifespan. The treated animal can be a healthy adult prior to treatment.

In one embodiment, the test compound modulates a metabolic process e.g. insulin signaling or oxidant scavenging. In an embodiment, the test compound regulates insulin signaling. In another preferred embodiment, the test compound modulates the effect of an environmental stress, e.g. the test compound is an anti-oxidant or the test compound activates superoxide dismutase.

In one embodiment, the first and second biological samples are obtained from the same specific tissue. For example, the specific tissue participates in a metabolic process. When the wildtype and mutant organisms of the second species are mammals (e.g. mouse), the tissue can be, for example, a tissue from liver, pancreas, pituitary, hypothalamus, or brain.

In another aspect, the method includes comparing expression of one or more genes in a reference animal to expression the one or more genes in a genetically distinct animal of the same species; and selecting a gene which is differentially expressed in the genetically distinct animal relative to the reference animal, provided that the reference animal and the genetically distinct animal are the same chronological age and the genetically distinct animal has an average lifespan at least 5, 10, 20, 40, 50, 80, or 100% greater than the reference animal. The method can include other features described herein.

In another aspect, the method includes comparing expression of one or more genes in a wildtype organism to expression the one or more genes in a genetically distinct organism of the same species; and selecting a gene which is differentially expressed, provided that the wildtype organism and the genetically distinct organism are the same chronological age and the genetically distinct organism senesces prematurely relative to the wildtype organism. The method can include other features described herein.

In another aspect, the invention features a method that includes: evaluating biomolecules in (a) a subject treated with a compound that reduces oxidative stress or provides anti-oxidant activity or (b) a sample obtained from the subject to obtain a subject-associated property for each of the biomolecules; comparing each subject-associated property to a corresponding reference property associated with a control subject to identify candidate biomolecules that have a statistically distinguishable property in the treated subject relative to the control subject; and identifying one or more of the candidate markers whose property is an indicator of an organism's lifespan. The method can include evaluating the respective property of each of the candidate molecules in genetically similar animals at different chronological ages; and identifying one or more of the candidate markers whose respective property is an indicator of chronological age. In another example, the method pertains to identifying by evaluating the respective property of each of the candidate molecules in a first and second animal at the same chronological age, wherein the genotype of the first animal is associated with a different average lifespan than the genotype of the second animal; and identifying one or more of the candidate markers whose respective property differs between the genetically-differing animals.

Compounds that provide antioxidant activity can include Vitamin E, Vitamin A, beta-carotene and other carotenoids, N-acetylcysteine and superoxide dismutase. In some examples, the compounds include manganese, e.g. manganese cyclan or MnDOTA.

In one embodiment, the treated subject is a mammal, e.g., a mouse, rat, primate, or human. In one embodiment, the treated subject and control subjected are exposed to an oxidative stress, e.g., a stress that elevates reactive oxygen species (ROS).

In some examples, the biomarker contains zinc or copper, or is associated with the presence of zinc or copper or the ratio of copper to zinc levels in tissues or organs (e.g., the brain). In other examples, the biomarker (e.g., a transcript or protein) is correlated with the presence of zinc or copper or the ratio therebetween.

The method also can include selecting a nucleic acid marker: providing a first nucleic acid population from a wildtype animal and a second nucleic acid population from a mutant animal, wherein the wildtype animal and the mutant animal are the same chronological age and the nucleic acid populations can include transcripts or cDNA replicates thereof evaluating the first and second nucleic acid populations using hybridization probes; and identifying a nucleic acid whose abundance in the first and second nucleic acid populations differs, thereby identifying a nucleic acid marker.

In another aspect of the invention, a database is disclosed that can include a plurality of records, each record including information indicating (a) identity of a biomolecule, (b) a property of the biomolecule in a subject organism, (c) genotype of the subject organism, and, optionally, (d) chronological age of the subject organism, wherein (1) the database includes records for at least two genotypes for organisms of the same species, the genotypes being associated with different expected lifespans, and (2) the database can be accessed to identify records for biomolecules that have different properties for genotypes associated with different expected lifespan. In one embodiment, the record further includes (e) information about exposure of the subject organism to a test compound.

In another aspect, the invention features a method that includes: providing a first organism having a first genotype and a second organism having the first genotype or a second genotype, provided that the second organism is subjected to conditions which target the function of at least one gene, wherein the first and second organisms are derived from the same species and are the same chronological age; and comparing a property associated with a biomolecule in the first organism to a property associated with the biomolecule in the second organism to identify a biomolecule having a preselected value for said property, thereby identifying the biomolecule as an biological age-associated marker. The marker, for example, can provide an indication of lifespan regulation in organisms derived from the particular species, and may be predictive of the potential lifespan of an individual. The second organism is subjected to conditions that target the function of one or more particular genes. For example, RNA interference, antisense RNA expression, and ribozymes can be used to target the one or more particular genes. These genes can be selected for the function in a particular pathway, e.g., the GH-IGF-1 axis, the SIR pathway, the Indy pathway, mitochondrial function, metabolic functions, the shc pathways, the oxidative stress response pathway and so forth. The targeted gene can be, for example, a gene described herein.

Methods of the invention can further includes comparing the profile to an expression profile of a reference sample, e.g., from an organism that does not include the non-wildtype or non-prevalent allele (e.g., is homozygous for the wildtype allele).

In another aspect, the invention features a computer medium having a plurality of digitally encoded data records. Each data record includes a value representing the level of expression of a particular protein or mRNA in a sample, and a descriptor of the sample. The descriptor of the sample can be an identifier of the sample, a subject from which the sample was derived (e.g., a particular strain, individual or patient with a lifespan disorder), or a treatment (e.g., a test compound). The data record can be structured as a table, e.g., a table that is part of a database such as a relational database (e.g., a SQL database of the Oracle or Sybase database environments).

The sample can be from a mutant worm, e.g., a daf mutant, a mutant mouse, e.g., a p66shc mutant, a mutant fly, e.g., an Indy mutant, and so forth.

Also featured is a computer medium having executable code for effecting the following steps: receive a query expression profile; access a database of reference expression profiles; and either i) select a matching reference profile most similar to the subject expression profile or ii) determine at least one comparison score for the similarity of the subject expression profile to at least one reference profile. The reference expression profiles represent a profile of a wildtype organism or sample thereof, or a mutant organism, e.g., a lifespan-affected mutant, or sample thereof.

In another aspect, the invention features a method of identifying a lifespan target. The method includes comparing a test profile to a reference profile (e.g., a reference profile above). In a preferred embodiment, the test profile is an expression profile of a mutant organism, e.g., a lifespan-affected mutant, e.g., a mutant that has extended or reduced lifespan relative to wildtype. The method includes identifying one or more mRNAs or proteins that are under- or over-expressed in the test profile. The identified MRNA or proteins are then used as targets, e.g., to identify a test compound that binds the identified mRNA or protein encoded by the MRNA, or the protein.

In another aspect, the invention features a method of identifying a target biomolecule (e.g., protein or RNA) that can modulate lifespan. The method includes determining test profiles for a mutant strain, as individuals of the strain age, clustering the genes in the test profiles, identifying biomolecules (i.e., mRNAs or proteins) that are coordinately regulated as the mutant organism ages. The identified biomolecules may be targets that regulate lifespan.

If a sufficient number of diverse samples is analyzed, clustering (e.g., hierarchical clustering, k-means clustering, Bayesian clustering and the like) can be used to identify other genes which are co-regulated during aging.

In another aspect, the invention features a method of assessing a test compound. The method includes: contacting a test compound to a cell or a subject; profiling the expression of biomolecules in the cell or subject; and comparing the profile to a reference profile, wherein the reference profile is the profile of a cell or subject that includes an allele of a gene associated with lifespan regulation.

In a preferred embodiment, genes that are associated with lifespan regulation can include DAF mutants, insulin pathway members (e.g., GH-IGF-1 pathway members), p66shc adaptors, a sir pathway members (e.g., SIR2), and shc pathway members, INDY pathway members, dicarboxylate transporters, and respiratory and oxidative pathway members.

A test compound that alters a profile of a cell or subject so as to be more similar to the reference profile of a lifespan regulation mutant that extends lifespan can be identified as a candidate compound for modulating lifespan.

In a preferred embodiment, test compound is an agonist or antagonist of a SIR protein or histone deacetylase, e.g., Sir2, an insulin pathway member, a dicarboxylate transporter, a respiratory or oxidative pathway member.

The term “chronological age” as used herein refers to time elapsed since a preselected event, such as conception, a defined embryological or fetal stage, or, more preferably, birth.

In contrast, the term “biological age” refers to phenotypic or physiological states that are not linearly fixed with the amount of time elapsed since a preselected event, such as conception, a defined embryological or fetal stage, or, more preferably, birth. The chronological age at which a phenotypic or physiological state occurs can vary between individuals. Exemplary manifestations of biological aging in mammals include endocrine changes (for example, puberty, menses, changes in fertility or fecundity, menopause, and secondary sex characteristics, such as balding, pubic or facial hair), metabolic changes (for example, changes in appetite and activity), and immunological changes (for example, changes in resistance to disease). The appearance of mammals also change with biological age, for example, graying of hair, wrinkling of skin, and so forth. With respect to a different class of animals, the nematode C. elegans also has manifestations of biological aging, for example, changes in fecundity, activity, responsiveness to stimuli, and appearance (e.g., change in intestinal autofluorescence and flaccidity). In many cases, the remaining potential lifespan of an individual is a function of its biological age.

The invention provides methods to discover and validate markers that distinguish biological age from chronological age. Methods of the invention are useful in a number of areas, including the discovery and validation of new targets for reducing rate of aging, extending life span, reducing incidence and delaying onset of disease and improving overall health of aging populations. Furthermore, the invention will facilitate the discovery and development of drugs, biologicals and treatment regimens based on the above that favorably intervene in the aging process. For example, markers identified by a method described herein can be used to choose target gene products in a therapeutic protocol, to elaborate the biological function of the target gene product in the aging process, and to identify compounds that alleviate deterioration associated with aging by modulating the activity of target gene products.

At least one particular advantage of many of the methods described herein is that a comparison is made between organisms of the same chronological age. The organisms differ by gene function, e.g., genotype. Thus, typically, changes that result from chronological age (e.g., accumulation of environmental exposure) are controlled for in both the organisms, particularly when the organisms can be maintained under controlled conditions. When biomolecules are compared between the two organisms, the detected differences in a property can be accurately attributed to their genotype, e.g., their differential rate of biological aging.

The details of one or more embodiments of the invention are set forth in the accompanying drawings and the description below. All patents, patent applications, and references cited herein are incorporated by reference in their entirety. Other features, objects, and advantages of the invention will be apparent from the description and drawings, and from the claims.

### DETAILED DESCRIPTION

The aging of living organisms includes complex developmental changes that occur over the passage of time. The invention is based, in part, on the observation that molecular mechanisms regulate the aging process. Thus, aging includes biologically programmed changes in addition to random or incremental accumulation of detrimental events that may result, for example, from exposure to the environment or stress. Furthermore, many of these programmed aging mechanisms may be conserved across species as diverse as yeast and humans. Modem molecular genetic techniques have enabled the discovery of conserved pathways that regulate lifespan in yeasts, nematodes, fruit flies and mice. In some cases, mutation in a single gene can result in altered lifespan (reviewed in, e.g., Guarente and Kenyon, Nature 2000; 408:255).

In at least one aspect, the invention provides for the identification of biomarkers which can have one or more of the following exemplary properties: (a) distinguish chronological age from biological age, (b) can be assayed with a non-invasive specimen (e.g., blood, urine, skin, saliva, etc.), (c) possess appropriate dynamic range across age spans of interest and (d) are conserved among distinct species. In one embodiment, candidate biomarkers are identified by comparing global gene expression of cells, tissues, organs and organisms among wild type and longevity gene mutant organisms at the same chronological ages. It is also possible to compare gene expression among model organisms with short life spans and simple genomes (yeast, flies, nematode worms) at different chronological ages. Candidate biomarkers can then be tested, e.g., in mice and humans, via transcriptional profiling of relevant cells, tissues and organs or in silico analyses of gene expression databases. In at least some cases, the process will lead to markers which in composite reliably distinguish chronological vs. biological age across the life span of an organism, e.g., a human or mouse, and possess one or more of the other desirable properties listed above and will be useful surrogates for judging efficacy of life span extending drug candidates.

The present invention provides a method for the identification of markers of aging. These markers (or “biomarkers”) are useful indicia of the developmental program in mature organisms. In one aspect of the invention, organisms of the same chronological age and of different genotypes are compared. Genetic variation can impact the biological aging process of each organism. Accordingly, the genotypes can be selected that result in different average lifespans. The term “average lifespan” refers to the average of the age of death of a cohort of organisms. In some cases, the “average lifespan” is assessed using a cohort of genetically identical organisms under controlled environmental conditions. Deaths due to mishap are discarded. For example, with respect to a nematode population, hermaphrodites that die as a result of the “bag of worms” phenotype are typically discard. Where average lifespan cannot be determined (e.g., for humans) under controlled environmental conditions, reliable statistical information (e.g., from actuarial tables) for a sufficiently large population can be used as the average lifespan. Characterization of molecular differences between two such organisms can reveal markers that correlate with the physiological state of the organisms. In some embodiments, the characterization is performed before the organisms exhibit overt physical features of aging. For example, the organisms may be adults that have lived only 10, 30, 40, 50, 60, or 70% of the average lifespan of a wildtype organism of the same species.

A variety of criteria can be used to determine whether organisms are of the “same” chronological age for the comparative analysis. Typically, the degree of accuracy required is a function of the average lifespan of a wildtype organism. For example, for the nematode C. elegans, for which the laboratory wildtype strain N2 lives an average of about 16 days under some controlled conditions, organisms of the same age may have lived for the same number of days. For mice, organism of the same age may have lived for the same number of weeks or months; for primates or humans, the same number of years (or within 2, 3, or 5 years); for Drosophila, the same number of weeks; and so forth. Generally, organisms of the same chronological age may have lived for an amount of time within 15, 10, 5, 3, 2 or 1% of the average lifespan of a wildtype organism of that species. In a preferred embodiment, the organisms are adult organisms, e.g. the organisms have lived for at least an amount of time in which the average wildtype organism has matured to an age at which it is competent to reproduce.

To identify a biomarker, a property associated with a candidate biomolecule in one organism is compared to the property of the corresponding biomolecule in the other organism. The “biomolecule” can be any molecule found in a biological sample or cell of the organism. Typically, such biomolecules are either identical to or derivatives of molecules that can be found in the organism. (e.g. cDNA is a derivative molecule). The term “biological sample” includes tissues, cells and biological fluids (e.g., serum, lymph, blood) isolated from an organism. In one aspect, the biological sample can be assayed with a non-invasive specimen (e.g. blood, urine, skin, saliva, etc.).

In one embodiment, the biomolecule is a nucleic acid molecule, which can include a DNA molecule (e.g. genomic DNA or cDNA generated from RNA), or RNA molecules (e.g. mRNA, tRNA, untranscribed RNAs). The nucleic acid molecule can be single-stranded or double-stranded. The nucleic acid molecule can be isolated or purified prior to analysis. If a nucleic acid molecule is identified as a biomarker, a variety of tools can be used to analyze subsequent samples. These tools include a probe or primer that is complementary to the nucleic acid molecule, a plasmid that includes the nucleic acid molecule, a host cell that can produce a protein encoded by the nucleic acid molecule, and a computer record that associates the nucleic acid molecule with a property corresponding to it in a particular sample. An isolated or purified nucleic acid molecule includes a nucleic acid molecule that is substantially free of other biomolecules present in the natural source of the nucleic acid. For example, a probe is an isolated nucleic acid molecule (although it may be present with other selected probes).

In another embodiment, the biomolecule is a protein (e.g., a polypeptide). An antibody or other ligand that specifically binds to the protein can be used to detect the protein. In many cases, a transcript which functions as a biomarker encodes a protein that is also a biomarker, and vice versa. In still other embodiments, the biomolecule is a polysaccharide (e.g. glucose, glycosaminoglycan), a lipid (e.g. phospholipid, sphingolipid, cholesterol), or other molecule, e.g., a metabolite, ligand which can bind metal ions (e.g., chelate) or other compound (e.g., superoxide).

To identify a biomarker, a property associated with a biomolecule in the first organism is compared to a property associated with the corresponding molecule in the second organism. In one embodiment, the property is abundance. Abundance of a biomolecule can be binary (e.g., present or absent), semi-quantitative (e.g., absent, low, medium, high), or quantitative. In another embodiment, the property is chemical composition. For example, with respect to protein biomolecules, this property can refer to post-translational modification state. Examples of post-translational modifications include glycosylation, phosphorylation, sulfation, ubiquitination, acetylation, lipidation, prenylation, and proteolytic cleavage. Modifications can be specific to a particular amino acid position in the protein. Chemical composition also includes substrate-product transformations. For example, a particular compound may be found in the first organism, but present in modified form (e.g., product) in the second organism. The property can also refer to enzymatic activity. For a biomolecule that is an enzyme, it may have certain catalytic parameters (e.g., K_{cat}, Kₘ, substrate specificity, allostery) in the first organism and other parameters in the second organism. In another embodiment, the property can be physical association with another biomolecule. In yet another embodiment, the property can refer to subcellular location of the biomolecule (e.g. ER, Golgi, cytosolic, nuclear, lysosomal, endosomal, plasma membrane, and extracellular matrix). Methods to evaluate these properties are described below or are known.

Generally, the property of the particular biomolecule is evaluated in the first and the second organisms. The respective properties are compared to determine if they have a preselected relationship. For example, for quantitative properties, they may differ by a preselected amount. The preselected amount can be any arbitrary value, and may not be known prior to the comparison, provided that the value is discrete and reproducible, e.g., for many comparisons of identical subjects or samples. Statistical significance can also be used to assess whether a preselected relationship is significant. Exemplary statistical tests include the Students T-test and log-rank analysis. Some statistically significant relationships have a P value of less than 0.05, or 0.02.

If the properties differ between the first and second organisms by a qualitatively or quantitatively detectable extent, then values (e.g., qualitative or quantitative values) are identified that are associated with the aging process. The value associated with the longer lived organism can be used as indication that the organism has a lifespan program that favors longevity, whereas the value associated with the shorter lived organism can be an indication that the organism has a lifespan program that does not support longevity to the extent of the longer lived organism.

Exemplary methods for evaluating biomolecules for the function as a marker of the aging process are described below and elsewhere herein.

Organisms

In one embodiment, the organism has a short average lifespan (e.g., less than 5, 3, or 2 years or less than 10, 6, or 1 month). The organism can be a model organism, e.g., a well characterized organism that can be breed and maintained under laboratory conditions. In addition, the model organism may also have a genome that is well characterized, e.g., genetically mapped and sequenced. Examples of such organisms include yeast (e.g., S. cerevisiae), flies (e.g., Drosophila), fish (e.g., zebrafish), nematodes (e.g., C. elegans and C. briggsae), and mammals (e.g., rodents (such as mice)).

As seen, biomarkers can be identified by of an organism of one genotype with an organism of a second genotype. As used herein, the term “genotype” refers to the genetic composition of an individual. The first and second genotypes can be two different naturally occurring genotypes. In another embodiment, the genotype of the first organism is wildtype and the genotype of the second organism is mutant. In still another embodiment, both genotypes are mutant. “Wildtype,” as used herein, refers to a reference genotype, including a genotype that predominates in a natural population or laboratory population of organisms as compared to natural or laboratory mutant forms. The lifespan phenotype of an average wildtype organism is necessarily a normal lifespan for the species.

An organism with a mutant genotype includes at least one genetic alteration, typically altering an endogenous gene of the organism. Such genetic alterations can be mapped. Examples of genomic alterations associated with mutant forms include point mutations, deletions, insertions, chromosomal rearrangements, transposon insertions, and retroviral insertions. In some particular embodiments, the genotype includes an alteration that results from an exogenous nucleic acid, e.g., a synthetic gene deletion construct, a transgene that inserted by recombination, an exogenous gene on an episome inserted by transformation, an exogenously introduced transposon or an exogenously introduced retroviral sequence. Genetic alterations can arise spontaneously; they can be present in a natural population at a low frequency (e.g., less than 5 or 2%); they can be generated in the laboratory (e.g., by exposure to mutagens or recombinant nucleic acids; see below).

Some exemplary genetic alterations occur in the genes listed in Table 1 and their homologs.

TABLE 1

| Organism | Gene name | Description | Exemplary homologs |
|---|---|---|---|
| S. cerevisiae | SIR2 | NAD-dependent histone | Murine Sir2alpha (GenBank AccNo: |
| | | deacetylase | AF214646), human SIRT1 (GenBank |
| | | | Acc No: AF083106) |
| | | | human Sir2 SIRT3 GenBank |
| | | | Accession No: AF083108; |
| | | | human Sir2 SIRT4 GenBank |
| | | | Accession No: AF083109; |
| | | | human Sir2 SIRT5 GenBank |
| | | | Accession No: AF083110 |
| | SIR3 | Regulator of chromatin silencing | |
| | SIR4 | Regulator of chromatin silencing | |
| | RPD3 | Histone deacetylase | |
| | FOB1 | Suppresses rDNA replication | |
| | SGS1 | Werners-like DNA helicase | |
| | SNF1 | Kinase involved in carbon source | |
| | | utilization | |
| | SIP2 | SNF1 co-repressor | |
| | SNF4 | SNF1 co-activator | |
| | NPT1 | Involved in NAD synthesis | |
| | RTG2 | Sensor of mitochondrial disfunction | |
| | Coq7 | Regulator of ubiquinone synthesis | |
| C. elegans | Daf-2 | Insulin/IGF-1 receptor homolog | insulin or IGF receptor |
| | Age-1 | PI(3) kinase | PI(3) kinase |
| | Pdk-1 | | PDK-1 |
| | Daf-18 | Phosphatase | PTEN |
| | Daf-16 | Forkhead/winged-helix family | AFX, FKHR, FKHRL1 |
| | | transcription factor | |
| | Ceinsulin-1 | Insulin/IGF-1-like homolog | insulin or IGF molecules |
| | Ctl-1 | Cytosolic catalase | |
| | MEV-1 | Cytochrome B subunit of | Cytochrome B subunit of |
| | | mitochondrial succinate | mitochondrial succinate |
| | | dehydrogenase | dehydrogenase |
| | Sod-3 | Mn-superoxide dismutase | superoxide dismutase |
| | Clk-1 | Regulator of ubiquinone synthesis | |
| | [Eat mutants] | | |
| | Tkr-1 | Tyrosine kinase | |
| | Spe-10 | Unknown (sperm defective) | |
| | Spe-26 | Unknown (sperm defective) | |
| | Old-1 | Receptor tyrosine kinase | |
| | Kin-29 | Serine Threonine Kinase | |
| Drosophila | Indy | Carboxylate transporter | hNaDC-1, accession No. U26209, |
| | GenBank accession | | SDCT2, accession no. AF081825, |
| | no. AE003519 | | NaDC-1, accession no. U12186, |
| | | | mNaDC-1, accession no. AF 201903, |
| | | | human solute carrier family 13, |
| | | | member 2 GenBank NP_003975.1, |
| | | | human sodium-dependent high- |
| | | | affinity dicarboxylate transporter 3, |
| | | | human carrier family 13 |
| | | | (sodium/sulfate symporters), member |
| | | | 1, human hypothetical protein |
| | | | XP_091606, human carrier family 13 |
| | | | (sodium/sulfate symporters) member |
| | | | 4 (GenBank NP_036582), |
| | Cu/Zn-SOD | superoxide dismutase | |
| | Methuselah | Putative G-protein-coupled 7 | |
| | | transmembrane domain receptor | |
| Mus musculus | p66shc | Signaling adaptor | |
| | PROP1 | Homeodomain protein | |
| | Growth hormone | | |
| | Growth hormone | | |
| | Releasing hormone | | |
| | receptor | | |

GH-IGF-1 Axis. Modulation of the growth hormone (GH)-insulin-like growth factor 1 (IGF-1) axis (also termed the GH-IGF-1 axis) may affection control of lifespan in man y organisms. For example, mutations in the insulin/IGF-1-like hormone receptor encoded by the daf-2 gene can double the lifespan of C. elegans (Kenyon et al. (1993) Nature 366(6454):461-4.). Mutations in other components of the GH-IGF-1 axis can similarly alter the lifespan of organisms. Examples of such components include:

hormones suchasaninsulin/IGF-1-like hormone, such as ceinsulin-1 and ceinsulin-1 homologs, mammalian insulin, mammalian IGF-1, somatostatin, growth hormone;

cell surface receptors (such insulin/IGF-1-like hormone receptor, GH releasing hormone (GHRH) receptor, GH receptor, and somatostatin receptors;

intracellular proteins that secrete GH or IGF-1 or the regulation the secretion; and proteins (intracellular and extracellular) that signal responses to GH, IGF-1, or somatostatin, e.g., a PI(3) kinase family member such as age-1 and age-1 homologs, pdk-1 and pdk-1 homologs, a Forkhead transcription factor such as daf-16 and daf-16 homologs which include AFX, FKHR, FKHRL1, and a PTEN phosphatase such as daf-18 and daf-18 homologs.

The second organism, for example, can include one or more genetic alterations that affect a gene or genes that encode a component of the GH-IGF-1 axis. A list of exemplary biomolecules includes: GHRF; GHRF-R; GH; GH-R; IGF-1; IGF-1R; PI(3)K; -p85; -p110; PTEN; PDK-1; AKT-1; AKT-2; AKT-3; PKCz; PKCl; FKHR; AFX; HNF1a; HNF1b; HNF4a; Insulin; INSII; Ins-R; IRS-1; IRS-2; IRS-3; IRS-4; UCP-1; UCP-2; UCP-3; UCP-4; p53; mclk1; socs2; and somatostatin.

Transcriptional Control. In another embodiment, the second genotype include one or more genetic alterations that affect a gene or genes that mediate transcriptional control, e.g., chromatin silencing, regulation of a nuclear protein such a transcription factor (e.g., p53), or regulation of histone acetylation state, e.g., the SIR2 pathway. For example, the gene may encode a protein that encodes a histone deacetylase. Examples of genes in which mutation can perturb regulation of such processes include in S. cerevisiae SIR4, SIR3, and SIR2, and homologs of these genes, e.g., genes encoding Murine Sir2 alpha (GenBank AccNo: AF214646), human SIRT1 (GenBank Acc No: AF083106), human Sir2 SIRT3 GenBank Accession No: AF083108, human Sir2 SIRT4 GenBank Accession No: AF083109, and human Sir2 SIRT5 GenBank Accession No: AF083 110. The substrate specificity of human Sir2 homologs can vary and may include diverse substrates, for example, nuclear substrates (e.g., p53), and cytoplasmic components (e.g., tubulin). The SIR2 pathway encompasses a network of proteins including, for example, RPD3 in yeast, and p53 in mammalian cells.

Metabolic Control. In another embodiment, the second genotype causes a defect in metabolic control. See, for example, regulation of the GH-IGF-1 axis above. Additional examples include metabolite sensing or metabolite transport. Examples of genes that are involved in metabolite sensing include genes encoding SNFI kinase, SIP2, a co-repressor of SNF-1, and SNF4, a coactivator of SNF1, clk-1, coq7, NPT1 and homologs of these proteins. Other relevant genes encode proteins that may participate in the transport of metabolites, e.g., the Indy transporter and other carboxylate transporters. Some such proteins may be mitochondrial membrane components.

Genes that indirectly participate in the metabolic sensing or other sensory processes may also affect lifespan control. For example, mutation of genes that affect neuronal cell fate can perturb sensation of various stimuli and thereby perturb lifespan control.

Oxidative Stress. In yet another embodiment, the second genotype causes a defect in genes that encode proteins that regulate the response to oxidative stress. Examples of proteins involved in the response to oxidative stress include catalases such as ctl-1, superoxide dismutases such as sod-3, succinate dehydrogenases such as mev-1, and certain signaling proteins, such as signaling adaptor components such as p66shc, spe-10, spe-26, old-1.

Additional exemplary genes that can affect lifespan control are described, for example, in Kenyon and Guarente, supra.

In another embodiment, the second genotype causes a defect in genes that involve endocrine signaling. More preferably, the gene is involved in growth hormone signaling, including growth hormone and pit-1/prop1.

In another embodiment, the second genotype is caused by a defect in a G-protein-coupled receptor. In a preferred embodiment, the G-protein-coupled receptor is Drosophila methuselah or a homolog of methuselah. In another embodiment, the genotype is caused by a mutation in the tyrosine kinase tkr-1 or a homolog of tkr-1.

In another embodiment, the genotype causes a defect in a mitochondrial component or a regulator of mitochondrial function. Mitochondrial functional is linked to at least some aging processes.

Other exemplary genes include: Tg2576; Klotho; pax3; Lep; Lepr; Pit1; Prop1; Sod1;

ApoE/A4App; Xrcc5/Ku86; Opg; Dmd/Utrn; Bdkrb2; Mpz Heterozygous/Gjb1 Homozygous; Spock; Hdh; G protein-coupled receptor G2A; Uteroglobin (Utg; Tgfb1; mito Sod2; Fas1; Telomerase RNA component (Terc; Acrb; Xrec5 homo/p53 hetero; ApoE/A4App; ApoE; Sam8 and others; and NOD.

Generation of Mutants

Generation of organisms with genetic alterations (e.g. transgenic, knockout) are well known in the art. For example, flies, nemotodes, and mice can be mutagenized with mutagens, crossed, and screened for mutant progeny. Mutations in existing animals can also be crossed into various other genetic backgrounds, e.g., to produce double mutants. In addition, molecular genetic methods can be used to generate, recover, and characterize genetic alterations. For example, once a gene of interest is known, it can be targeted by such molecular genetic methods and also by classical methods, e.g., saturation mutagenesis.

For Drosophila, P-element insertion can be used (E. Bier et al., Genes Dev. 3, 1273-1287 (1989); Spradling et al., Science, 218, 341-347 (1982)) and screened for a desirable trait. For example, flies that outlive the parent strain may be selected in a screen for mutants with alterations in lifespan. For C. elegans, Tc1 transposition, chemical mutagenesis with agents such as ethyl methanesuphonate or psoralen or UV can be used to produce genetic alterations.

For mice, one method for producing a transgenic mouse in which a specific site in the genome has been disrupted is as follows. Briefly, a targeting construct which is designed to integrate by homologous recombination with the endogenous nucleic acid sequence in the genome is introduced into embryonic stem cells (ES). The ES cells are then cultured under conditions that allow homologous recombination (i.e., of the recombinant nucleic acid sequence of the targeting construct and the genomic nucleic acid sequence of the host cell chromosome). ES cells identified as containing a recombinant allele are introduced into an animal at an embryonic stage using standard techniques which are well known in the art (e.g., by microinjection into a blastocyst). The resulting chimeric blastocyst is then placed into the uterus of a pseudo-pregnant foster mother for the development into viable pups. The resulting offspring include potentially chimeric founder animals whose somatic and germline tissue can contain a mixture of cells derived from the genetically-engineered ES cells and the recipient blastocyst. If the genetically altered stem cells have contributed to the germline of the resulting chimeric animals, the altered ES cell genome containing the disrupted target genomic locus can be transmitted to the progeny of these founder animals thereby facilitating the production of genetically altered animals.

It is also possible to use other technologies to reduce gene function. These include anti-sense, RNA interference, and ribozyme-mediated cleavage. In such embodiments, gene function is reduced without altering a genotype in a second organism.

Methods of Identifying Biomolecular Markers

A variety of methods can be used to identify biomolecular markers that are associated with aging or lifespan regulation. Typically, a plurality of biomolecules are evaluated for the first and second organism. The property of each biomolecule is identified in the respective organisms Properties that are detectably different identify the particular biomolecule as a marker, or at least a candidate biomarker.

Nucleic Acid Markers

In many embodiments, transcripts are analyzed from the two organisms. One method for comparing transcripts uses nucleic acid microarrays that include a plurality of addresses, each address having a probe specific for a particular transcript. Such arrays can include at least 100, or 1000, or 5000 different probes, so that a substantial fraction, e.g., at least 10, 25, 50, or 75% of the genes in an organism are evaluated. mRNA can be isolated from a sample of the organism or the whole organism. The mRNA can be reversed transcribed into labeled cDNA. The labeled cDNAs are hybridized to the nucleic acid microarrays. The arrays are detected to quantitate the amount of CDNA that hybridizes to each probe, thus providing information about the level of each transcript.

Methods for making and using nucleic acid microarrays are well known. For example, nucleic acid arrays can be fabricated by a variety of methods, e.g., photolithographic methods (see, e.g., U.S. Pat. Nos. 5,143,854; 5,510,270; and. 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated mRNA from samples for comparison can be reversed transcribed and optionally amplified, e.g., by rtPCR, e.g., as described in (U.S. Pat. No. 4,683,202). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

A general scheme for producing and evaluating profiles can include the following. The extent of hybridization at an address is represented by a numerical value and stored, e.g., in a vector, a one-dimensional matrix, or one-dimensional array. The vector x has a value for each address of the array. For example, a numerical value for the extent of hybridization at a first address is stored in variable xₐ. The numerical value can be adjusted, e.g., for local background levels, sample amount, and other variations. Nucleic acid is also prepared from a reference sample and hybridized to an array (e.g., the same or a different array), e.g., with multiple addresses. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different nucleic acids detected by the array.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Other methods for quantitating nucleic acid species include: quantitative RT-PCR. In addition, two nucleic acid populations can be compared at the molecular level, e.g., using subtractive hybridization or differential display.

In addition, once a set of nucleic acid transcripts are identified as being associated with aging or lifespan regulation, it is also possible to develop a set of probes or primers that can evaluate a sample for such markers. For example, a nucleic acid array can be synthesized that includes probes for each of the identified markers.

Protein Analysis

The abundance of a plurality of protein species can be determined in parallel, e.g., using an array format, e.g., using an array of antibodies, each specific for one of the protein species. Other ligands can also be used. Antibodies specific for a polypeptide can be generated by known methods.

Methods for producing polypeptide arrays are described, e.g., in De Wildt et al., (2000) Nature Biotech. 18:989-994; Lueking et al., (1999) Anal. Biochem. 270:103-111; Ge, H. (2000) Nucleic Acids Res. 28:e3, I-VII; MacBeath and Schreiber, (2000) Science 289, 1760-1763; Haab et al., (2001) Genome Biology 2(2):research0004.1; and WO 99/51773A1. A low-density (96 well format) protein array has been developed in which proteins are spotted onto a nitrocellulose membrane Ge, H. (2000) Nucleic Acids Res. 28, e3, I-VII). A high-density protein array (100,000 samples within 222×222 mm) used for antibody screening was formed by spotting proteins onto polyvinylidene difluoride (PVDF) (Lueking et al. (1999) Anal. Biochem. 270, 103-111). Polypeptides can be printed on a flat glass plate that contained wells formed by an enclosing hydrophobic Teflon mask (Mendoza, et al. (1999). Biotechniques 27, 778-788.). Also, polypeptide can be covalently linked to chemically derivatized flat glass slides in a high-density array (1600 spots per square centimeter) (MacBeath, G., and Schreiber, S. L. (2000) Science 289, 1760-1763). De Wildt et al., describe a high-density array of 18,342 bacterial clones, each expressing a different single-chain antibody, in order to screening antibody-antigen interactions (De Wildt et al. (2000). Nature Biotech. 18, 989-994). These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address and analyze similar to nucleic acid arrays.

Mass Spectroscopy. Mass spectroscopy can also be used, either independently or in conjunction with a protein array or 2D gel electrophoresis. For 2D gel analysis, purified protein samples from the first and second organism are separated on 2D gels (by isoelectric point and molecular weight). The gel images can be compared after staining or detection of the protein components. Then individual “spots” can be proteolyzed (e.g., with a substrate-specific protease, e.g., an endoprotease such as trypsin, chymotrypsin, or elastase) and then subjected to MALDI-TOF mass spectroscopy analysis. The combination of peptide fragments observed at each address can be compared with the fragments expected for an unmodified protein based on the sequence of nucleic acid deposited at the same address. The use of computer programs (e.g., PAWS) to predict trypsin fragments, for example, is routine in the art. Thus, each address of spot on a gel or each address on a protein array can be analyzed by MALDI. The data from this analysis can be used to determine the presence, abundance, and often the modification state of protein biomolecules in the original sample. Most modifications to proteins cause a predictable change in molecular weight.

Other methods. Other methods can also be used to profile the properties of a plurality of protein biomolecules. These include ELISAs and Western blots. Many of these methods can also be used in conjunction with chromatographic methods and in situ detection methods (e.g., to detect subcellular localization).

Other Biomolecules

Other biomolecules (e.g., other than proteins and nucleic acids) can be detected by a variety of methods include: ELISA, antibody binding, mass spectroscopy, enzymatic assays, chemical detection assays, and so forth.

Marker Orthologs

When a particular biomolecule is identified as a useful biomarker, e.g., because of at least one of its associated properties, it is also possible to identify its orthologs in other species, e.g., in mammalian species such as mice, rats, dogs, cows, pigs, primates, and human. Typically an “ortholog” is the closest homolog in a particular species to the biomolecule of interest such that the ortholog has in common at least one featured function of the biomolecule of interest. Orthologs are more easily identified when complete or partially complete genome sequence is available for the organism, although PCR, hybridization, and EST analysis methods can substitute.

Homology can be determined by a number of routine methods. For example, the comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid biomolecule of interest. BLAST protein searches can be performed with the XBLAST program, score=50, wordlength=3 to obtain amino acid sequences homologous to protein biomolecule of interest. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Databases and Profiles

Also featured is a method of evaluating a sample and determining a profile of the sample, wherein the profile includes a value representing the level of biomolecules or other properties associated with biomolecules. In one embodiment, a profile of a sample from an organism that includes a non-wildtype, or a non-prevalent allele of a gene can be included. In a more preferred embodiment, the allele causes the organism to have increased or decreased lifespan. As used herein, “profile” refers to a set of values or qualitative descriptors, each value or descriptors, each value or descriptor representing the level of expression (protein or mRNA) of a particular gene. The organism can be a metazoan, e.g., a mammal (e.g., a mouse, rat, dog, or human), or an invertebrate, e.g., a fly.

In some embodiments, the profile is determined by contacting the sample or molecules extracted or amplified from the sample to a nucleic acid array. In another embodiment, the profile is determined by contacting the sample or molecules extracted from the sample to a protein array. In still another embodiment, the profile is determined by mass spectroscopy. The method can further relate to comparing the value or the profile (i.e., multiple values) to a reference value or reference profile. The profile of the sample can be obtained by any of the methods described herein (e.g., by providing a nucleic acid from the sample and contacting the nucleic acid to an array). The method can be used to monitor a treatment e.g., a subject treated with a test compound or an approved therapeutic. For example, the gene expression profile can be determined for a sample from a subject undergoing treatment with a test compound. In a preferred embodiment, the method further includes comparing the profile to an expression profile of a reference sample, e.g., from an organism that does not include the non-wildtype or non-prevalent allele (e.g., is homozygous for the wildtype allele).

In one aspect, the invention provides for a computer medium having a plurality of digitally encoded data records. For example, each data record includes a value representing the level of expression of a biomolecule in a sample, and a descriptor of the sample. The descriptor of the sample can be an identifier of the sample, a subject from which the sample was derived (e.g., an organism such as a mouse), a treatment (e.g., a treatment with a test compound). In a preferred embodiment, the data record further includes values representing the level of expression of additional biomolecules (e.g., other genes or proteins associated with aging, or other genes on an array). The data record can be structured as a table, e.g., a table that is part of a database such as a relational database (e.g., a SQL database of the Oracle or Sybase database environments).

The sample can be from an animal with a genotype that causes an alteration in lifespan regulation relative to the norm, e.g., a mutant worm, e.g., a C. elegans daf mutant, a mutant mouse, e.g., a p66shc mutant, an Ames or Snell mouse, a mutant fly, e.g., an Indy mutant and so forth.

Also featured is a computer medium having executable code for effecting the following steps: receive a query expression profile; access a database of reference expression profiles; and either i) select a matching reference profile most similar to the subject expression profile or ii) determine at least one comparison score for the similarity of the subject expression profile to at least one reference profile. The reference expression profiles can represent a profile of a wildtype organism or sample thereof, or a mutant organism, e.g., a lifespan-affected mutant, or sample thereof.

The computer-based techniques described here are not limited to any particular hardware or software configuration; they may find applicability in any computing or processing environment. The techniques may be implemented in hardware, software, or a combination of the two. For example, the techniques can be implemented using embedded circuits. Computer-based techniques may be implemented in programs executing on programmable machines such as mobile or stationary computers, handheld devices, biological sample handling or sensing apparati, and similar devices that each include a processor, a storage medium readable by the processor (including volatile and non-volatile memory and/or storage elements), at least one port or device for video input, and one or more output devices (e.g., for video storage and/or distribution).

An example of a programmable system, suitable for implementing a described video encoding method, includes a processor, a random access memory (RAM), a program memory (for example, a writable read-only memory (ROM) such as a flash ROM), a hard drive controller, and an input/output (I/O) controller coupled by a processor (CPU) bus. The system can be preprogrammed, in ROM, for example, or it can be programmed (and reprogrammed) by loading a program from another source (for example, from a floppy disk, a CD-ROM, or another computer). The hard drive controller is coupled to a hard disk suitable for storing executable computer programs and/or encoded video data. The I/O controller is coupled to an I/O interface. The I/O interface receives and transmits data in analog or digital form over a communication link e.g., a link to a local area network, a virtual private network, or the Internet.

Programs may be implemented in a high-level procedural or object oriented programming language to communicate with a machine system. However, the programs can be implemented in assembly or machine language, if desired. In any case, the language may be a compiled or interpreted language. Each such program may be stored on a storage medium or device, e.g., compact disc read only memory (CD-ROM), hard disk, magnetic diskette, or similar medium or device, that is readable by a general or special purpose programmable machine for configuring and operating the machine when the storage medium or device is read by the computer to perform the procedures described in this document. The system may also be implemented as a machine-readable storage medium, configured with a program, where the storage medium so configured causes a machine to operate in a specific and predefined manner.

Target Identification and Validation

Many methods of target identification and validation utilize molecular-genetics (forward and reverse genetics) and biochemical (e.g., RNAi, antisense, target-specific antibody, other target binding ligands) approaches in model organisms, including yeast, flies, nematode worms, and mice to identify genes which when perturbed extend life span. Through access to human population genetics, candidate genes identified in the model organisms can be validated, e.g., via association analyses. In addition, novel human gene associated with extended life span can be identified via association analyses (e.g., positional cloning).

Methods which can be employed include:

1. in silico analysis of EST, gene expression, protein-protein interaction, biochemical-metabolic pathway, structure-function, and other genetic-function databases can be used to accomplish one or more of the following: (1) identify candidate human orthologs of longevity genes identified in model organisms, (2) obtain tissue and developmental expression information for candidate genes, (3) identify potential polymorphisms associated with candidate genes which may be associated with human longevity phenotypes, (4) assign encoded proteins to pathways, (5) identify other molecular participants in these pathways, (6) construct structural models for encoded proteins, (6) establish function(s) and mechanisms of action, (7) identify compounds known to interact with members of the pathway and access pharmacological, structural, and other information for those compounds, and (8) relationship(s) of members of pathways to specific diseases.

2. transcriptional profiling of gene expression in cells, tissues, organs, and organisms can be used to accomplish one or more of the following: (1) assess effect of genetic and/or biochemical perturbation of longevity genes on global gene expression in model organisms and humans through early development, maturation and aging, (2) measure tissue and developmental expression of longevity genes, members of longevity pathways and genes effected by perturbing longevity genes, (3) global comparisons of gene expression in model organisms with short life span and simple genomes (e.g., yeast, nematode worms, flies) comparing different chronological ages to identify potential longevity genes, (4) determine mechanism(s) of action, potential toxicities and identify target(s) of compounds obtained from longevity screens, (5) global assessments of gene expression among organisms of different chronological and biological ages to identify potential targets and pathways for pharmacological intervention.

3. construct transgenic animal models in which candidate longevity genes, e.g., genes that are involved in mitochondrial function or energy metabolism (e.g., transporter molecules), heat shock response, insulin signaling, or, and/or designed mutants of candidate longevity genes are incorporated to achieve controlled expression (e.g., quantitative control as well as developmental, tissue, etc.) in the organism.

Assays that can be used include methods for assessing the expression level of biomolecules and for identifying variations between such molecules in organisms of different genotypes. Detailed examples of such assays are provided herein.

Evaluating a Test Compound

Embodiments include carrying out primary compound screens for life span extension in vitro using molecular or cell-based assays and/or in vivo using simple model organisms with automated, high throughput, high capacity screens. Surrogate life span markers (see above) can replace measuring death as an assay endpoint for the in vivo screens, and therefore speed these screens. Positives from these primary screens can then be assayed in an animal, e.g., a fly, worm, or mouse, and actual life span can be measured for animals treated with one of a smaller number of compounds at this stage, although, here again, reliable life span surrogate markers for the organism can be used as well. Transcriptional profiling can be used to assess efficacy, mechanism of action, potential toxicity and pharmacogenetic features of candidate life span extending compounds which emerge from our screens. As described above (see “Target Identification and Validation”), transcriptional profiling can also identify potential targets for those compounds derived from cell-based and in vivo screens. Test compounds can be evaluated using animal models, particularly mice, where we have previously identified markers for life span extension efficacy, as described above, often based on information gleaned from the simpler model organisms.

In one aspect, the invention provides assays for screening for a test compound, or more typically, a library of test compounds, to evaluate an effect of the test compound on an age-related process. The method includes contacting a system such as a cell or an organism with the test compound and evaluating a property of a marker that is associated with lifespan regulation or the aging process. The property can be compared to a control system, e.g., to see if the test compound perturbs the system relative to the control system which is not exposed to the test compound and which is typically maintained under otherwise identical conditions. A test compound that causes a change in a property of a biomarker so that the property moves towards or adopts characteristics of subject have genotypes associated with longevity may identify the test compound as a compound that can prolong longevity. The test compound may also be considered a lead compound that is further modified and optimized. Modified forms can be similarly assayed. In another example, a test compound that causes a change in a property of a biomarker so that the property moves towards or adopts characteristics of a subject that has a genotype associated with reduced lifespan may identify the test compound as a compound that alters lifespan regulation to reduce lifespan. Such a test compound may be modified or redesigned to favorably modulation lifespan regulation. For example, redesign can turn certain agonists into antagonists and vice versa. In addition such a test compound can be used as an entry point to identify a target molecule for which other regulators be targeted.

At least one advantage of evaluating the marker rather than lifespan itself is speed. For example, the system does not need to be maintained for the full lifespan of the organism. Typically, the cell or organism is exposed to the test compound, and after an interval (e.g., a few hours, or days), the cell or organism is characterized, e.g., for a biomarker associated with again. In addition, the test compound can be contacted to cells and organisms at different ages to evaluate an age-based response. Third, the assays can be done without a particular direct target in mind.

A “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., a herb or a nature product), synthetic, or both. Examples of macromolecules are proteins, protein complexes, and glycoproteins, nucleic acids, e.g., DNA, RNA and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heteroorganic or organometallic compounds. A test compound can be the only substance assayed by the method described herein. Alternatively, a collection of test compounds can be assayed either consecutively or concurrently by the methods described herein.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al (1994) J. Med. Chem. 37:1233.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The test compounds of the present invention can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. BioL 222:301-310; Ladner supra.).

In yet another aspect, the invention features a method of evaluating a test compound using a plurality of biomarkers. This can be done by profiling the sample. The method includes providing a cell and a test compound; contacting the test compound to the cell; obtaining a subject expression profile for the contacted cell; and comparing the subject expression profile to one or more reference profiles. The profiles include a value representing the level of expression of molecules previously determined to be involved in age-related processes. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

Similarity of profiles can be determined by a variety of metric, including Euclidean distance in a n-dimensional space, where n is the number of different values within the profile. Other metrics, for example, include weighting factors that basis different values according to their importance for the comparison.

Profiles, e.g., profiles obtained from nucleic acid array or protein arrays can be used to compare samples and/or cells in a variety of states as described in Golub et al. ((1999) Science 286:531). In one embodiment, multiple expression profiles from different conditions and including replicates or like samples from similar conditions are compared to identify nucleic acids whose expression level is predictive of the sample and/or condition. Each candidate nucleic acid can be given a weighted “voting” factor dependent on the degree of correlation of the nucleic acid's expression and the sample identity. A correlation can be measured using a Euclidean distance or the Pearson correlation coefficient.

Diagnostics and Patient Care

The biomarkers identified by the method described herein can also be used for diagnostic purposes, e.g., in patient care. For example, the markers can be used in a method of evaluating a subject. The subject can be a healthy or affect subject, e.g., an adult patient or a patient undergoing treatment. An exemplary method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of expression of molecules identified as markers for aging. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of expression of markers for aging.

Reactive Oxygen Species

Biological tissues can be damaged by a variety of stresses, including oxidative stress which can contribute to aging and degenerative diseases (e.g., amyothrophic lateral sclerosis). Exemplary reactive oxygen species include oxygen radicals (e.g., superoxide), and hydrogen peroxide. Collectively these are termed reactive oxygen species (ROS). Many free radical reactions are highly damaging to cellular components; they can crosslink proteins, mutagenize DNA, and peroxidize lipids.

In one embodiment, a cell or organism is treated with an agent that mitigates or is suspected of mitigating the environmental stress. For example, with respect to ROS, exemplary agents include synthetic catalytic scavenger compounds and agents which activate or otherwise increase activity of superoxide dismutase or catalase. Exemplary ROS binding compounds include homocystine, clioquinol, and diaminodicarboxylate. Still other compounds are described in U.S. Pat. Nos. 5,403,834, 5,696,109, 5,827,880, 5,834,509 and 6,046,188 describing a salen-transition metal complex, e.g., a salen-Mn(III) complex that is a free radical scavenger.

The cell or organism is evaluated to identify a biomarker that is associated with the mitigating effects of the agent. Such a biomarker is useful, e.g., to identify natural or artificial compounds that have a similar effect as the agent.

In one example, the biomarker is a biomolecule that contains copper or zinc. Further, it is possible to evaluate the concentrations of Cu and Zn in brain tissue over the lifespan of an animal or in animals (e.g., mammals) of different genotypes at the same chronological age. Evaluating biomolecules that correlate with concentration of Cu or Zn identifies markers that can be used to detect physiological states associated with high concentrations of these elements, as occurs in certain disorders (e.g., Alzheimer's disease).

A number of embodiments of the invention have been described. Nevertheless, it will be understood that various modifications may be made without departing from the spirit and scope of the invention. Accordingly, other embodiments are within the scope of the following claims.

What is claimed is:

1. A method of identifying an biological age-associated marker, the method comprising:
providing a first organism having a first genotype and a second organism having a second genotype, wherein the first and second organisms are derived from the same species and are the same chronological age; and
comparing a property associated with a biomolecule in the first organism to a property associated with the biomolecule in the second organism to identify a biomolecule having a preselected value for said property, thereby identifying the biomolecule as an biological age-associated marker.

2. The method of claim 1 wherein a plurality of properties associated with the biomolecule are compared.

3. The method of claim 1, wherein the comparing comprises providing a first biological sample from the first organism and a second biological sample from the second organism and evaluating the property of the biomolecule in the respective biological samples.

4. The method of claim 1, wherein the comparing is repeated for a property of each of a plurality of biomolecules.

5. The method of claim 3, wherein the biomolecules comprise nucleic acids.

6. The method of claim 3, wherein the biomolecules comprise proteins.

7. The method of claim 1, wherein the property is abundance.

8. The method of claim 1, wherein the property is chemical composition of the biomolecule.

9. The method of claim 6, wherein the property is a post-translational modification.

10. The method of claim 1, wherein the property is functional activity.

11. The method of claim 10, wherein the functional activity is assessed in the presence of a reactive oxygen species (ROS).

12. The method of claim 1, wherein the property is subcellular distribution.

13. The method of claim 1, wherein the property is physical association with another biomolecule.

14. The method of claim 5, wherein the comparing comprises hybridization to a nucleic acid array.

15. The method of claim 5, wherein the comparing comprises nucleic acid tag analysis.

16. The method of claim 4, wherein a plurality of markers are identified, the plurality being a subset of the plurality of biomolecules.

17. The method of claim 4, wherein the comparing comprises evaluating the respective sample to provide a sample profile that comprises information about one or more properties for each of a plurality of candidate markers, storing information about the profile in a machine-accessible medium, evaluating statistical significance of differences between corresponding candidate markers, and displaying information that identifies a subset of the candidate markers for which the differences are statistically significant.

18. The method of claim 1, wherein the first and second organisms are invertebrates.

19. The method of claim 1, wherein the first and second organisms are vertebrates.

20. The method of claim 1, wherein the first genotype is a wildtype genotype, and the second genotype is a mutant genotype.

21. The method of claim 20, wherein the second, mutant genotype is characterized by altered lifespan relative to the wildtype genotype.

22. The method of claim 21, wherein the altered lifespan is lifespan extension.

23. The method of claim 21, wherein the altered lifespan is lifespan reduction.

24. The method of claim 1, wherein the second genotype comprises homozygous mutations in two genes that each independently alter lifespan.

25. The method of claim 1, wherein the first genotype is a mutant genotype, and the second genotype is a mutant genotype.

26. The method of claim 1, wherein the first genotype causes lifespan extension relative to wildtype organisms of the same species and the second genotype causes lifespan reduction relative to wildtype organisms of the same species.

27. The method of claim 1, wherein the chronological age is an adult age.

28. The method of claim 1, wherein the chronological age is between 50% and 75% of the average lifespan of the first organism.

29. The method of claim 1, wherein the second organism has an average lifespan that is at least 20% greater than the average lifespan of the first organism.

30. The method of claim 1, wherein the second organism has an average lifespan that is at least 25% greater than the average lifespan of wildtype organisms of the same species.

31. The method of claim 1, wherein the second organism has an average lifespan that is at least 25% less than the average lifespan of wildtype organisms of the same species.

32. The method of claim 1, wherein the second genotype causes a defect in a growth hormone or insulin-like growth factor signaling component.

33. The method of claim 1, wherein the comparing is repeated at multiple chronological ages.

34. The method of claim 3, wherein the biological samples comprise a mixture of purified proteins.

35. The method of claim 1, further comprising: selecting, from biomolecules of a second animal species, an ortholog of the identified marker, and evaluating one or more properties of the ortholog in an organism of the second species.

36. The method of claim 35, wherein the evaluating comprises evaluating the property of the ortholog in genetically-identical organisms of the second species, the organisms being of a differing chronological age.

37. The method of claim 3, further comprising evaluating a property of the marker in a third biological sample.

38. The method of claim 37, wherein the third biological sample is obtained from cultured cells treated with a test compound.

39. The method of claim 37, wherein the third biological sample is obtained from an animal treated with a test compound.

40. The method of claim 39, wherein the treated animal is treated with the test compound for less than 25% of its average lifespan.

41. The method of claim 1, wherein the property of the identified biomolecule is abundance and the preselected value corresponds to at least a 2 fold difference in the property.

42. The method of claim 3, wherein the first and second biological samples are obtained from the same specific tissue.

43. A method of selecting a marker, the method comprising:
comparing expression of one or more genes in a reference animal to expression of one or more genes in a genetically distinct animal of the same species; and
selecting a gene which is differentially expressed in the genetically distinct animal relative to the reference animal, provided that the reference animal and the genetically distinct animal are the same chronological age and the genetically distinct animal has an average lifespan at least 20% greater than the reference animal.

44. A method of selecting a marker, the method comprising:
comparing expression of one or more genes in a wildtype organism to expression of the one or more genes in a genetically distinct organism of the same species; and
selecting a gene which is differentially expressed, provided that the wildtype organism and the genetically distinct organism are the same chronological age and the genetically distinct organism senesces prematurely relative to the wildtype organism.

45. A method of identifying a biomarker, the method comprising:
evaluating biomolecules in (a) a subject treated with a compound that alters response to an environmental stress or (b) a sample obtained from the treated subject to obtain a subject-associated property for each of the biomolecules;
comparing each subject-associated property to a corresponding reference property associated with a control subject to identify candidate biomolecules that have a statistically distinguishable property in the treated subject relative to the control subject; and
identifying one or more of the candidate markers whose respective properties are an indicator of an organism's lifespan.

46. The method of claim 45, wherein the agent mitigates oxidative stress.

47. The method of claim 45, wherein the identifying comprises:
evaluating the respective property of each of the candidate molecules in genetically similar animals at different chronological ages; and
identifying one or more of the candidate markers whose respective property is an indicator of chronological age.

48. The method of claim 45, wherein the identifying comprises:
evaluating the respective property of each of the candidate molecules in a first and second animal at the same chronological age, wherein the genotype of the first animal is associated with a different average lifespan than the genotype of the second animal; and
identifying one or more of the candidate markers whose respective property differs between the genetically-differing animals and is an indicator of biological age

49. The method of claim 46, wherein the compound is selected from the group consisting of: Vitamin E, Vitamin A, beta-carotene, and N-acetylcysteine.

50. The method of claim 46, wherein the compound activates superoxide dismutase.

51. The method of claim 46, wherein the compound contains manganese.

52. A method of selecting a nucleic acid marker, the method comprising:
providing a first nucleic acid population from a wildtype animal and a second transcript population from a mutant animal, wherein the wildtype animal and the mutant animal are the same chronological age and the nucleic acid populations comprises transcripts or cDNA replicates thereof;
evaluating the first and second nucleic acid populations using hybridization probes; and
identifying a nucleic acid whose abundance in the first and second nucleic acid populations differs, thereby identifying a nucleic acid marker.

53. A database comprising a plurality of records,
each record comprising information indicating (a) identity of a biomolecule, (b) a property of the biomolecule in a subject organism, (c) genotype of the subject organism, and, optionally, (d) age of the subject organism,
wherein (1) the database comprises records for at least two genotypes for organisms of the same species, the genotypes being associated with different expected lifespans, and
(2) the database can be accessed to identify records for biomolecules that have different properties for genotypes associated with different expected lifespan.

54. The database of claim 53, wherein the record further comprises (e) information about exposure of the subject organism to a test compound.
